(19) United States
(12) Patent Application Publication
(10) Pub. No.: US 2007/0027451 A1
(43) Pub. Date: Feb. 1, 2007

(54) Method for treatment of hypertrophic palatine tonsils

(76) Inventors: Kai Desinger, Berlin (DE); Andre Roggan, Berlin (DE); Christoph Janott, Berlin (DE); Fahri Yildiz, Koln (DE)

Correspondence Address: BECK AND TYSVER P.L.L.C., 2900 THOMAS AVENUE SOUTH, SUITE 100, MINNEAPOLIS, MN, 55416, US

(21) Appl. No.: 11/473,786

(22) Filed: Jun. 23, 2006

Related U.S. Application Data
(60) Provisional application No. 60/693,359, filed on Jun. 23, 2005.

(51) Int. Cl. A61B 18/14

(52) U.S. Cl. 606/50

(57) ABSTRACT

A method and apparatus are taught in which tonsils are be treated by using an applicator in a bipolar design. This means that no neutral electrode (i.e. no separate return electrode) is necessary.

This application claims the benefit of U.S. Ser. No. 60/693,359, filed Jun. 23, 2005.

### BACKGROUND OF THE INVENTION

This invention relates to a method of treatment of hyperplasic palatine tonsils.

Hyperplasic palatine tonsils can cause a series of complications. Especially children often display disturbances in growth, development and non-specific behavioral attributes such as hyperactivity, bed-wetting and increased irritability. Characteristic Symptoms of hyperplasia of the palatine tonsils may also be dysphagia and language disorders.

### SUMMARY OF THE INVENTION

It is an object of the invention to provide a method of treating hyperplasic palatine tonsils that is minimal invasive and reliable.

According to the invention this object is achieved by treating tonsils using an applicator in a bipolar design.

The applicator to be used has a hand portion and a shaft connected together. The shaft has at least two electrodes which are arranged on the shaft and which are spaced apart from each other in axial direction of the shaft and are insulated from each other by an insulator. An insulation tube insulates a portion of the electrode closest to the hand portion. Both electrodes are electrically. The electrode farthest from the hand portion has a conical shape tip to penetrate the tonsil tissue easily.

The treatment includes the steps of

a) pre-operative pain reduction,
b) superficial disinfection of the tonsils,
c) local anesthesia of the tonsil or general anesthesia,
d) treating the tonsils using a bipolar applicator having two electrodes coaxially arranged on a common shaft and spaced apart from each other in the longitudinal direction of the shaft,
e) peri-operative antibiotic prophylaxis, and
f) post-operative pain reduction.
   The use of said bipolar applicator according to step d) includes the steps of
1) connecting the applicator with a high frequency generator;
2) setting the power Output of the high, frequency generator to a value between 1 to 25 W, preferably 7 watts;
3) inserting the distal applicator tip into a tonsil to be treated until the insulation tube on the applicator shaft contacts the tonsil tissue and both electrodes are completely covered by the tonsil tissue;
4) activating the high frequency current in order to create a thermal lesion until the high frequency generator indicates the end of treatment by a pulsed acoustic signal, meaning that the dehydration of the tonsil tissue has been reached;
5) retracting the applicator tip from the tonsil; and
6) repeating the steps of Inserting, Activating and Retracting at different positions of the tonsil until a major part of the tonsil tissue is coagulated.
   Further preferred embodiments are illustrated in the following detailed description of the invention.

### BRIEF DESCRIPTION OF THE DRAWINGS

FIG. 1 is a plan view of the apparatus of the present invention.

FIG. 2 shows the electrode of the present invention in use treating tonsils.

FIG. 3 shows an enlarged longitudinally cut view of a distal portion the shaft.

FIG. 4 illustrates a treatment of tonsils using an applicator as depicted in FIGS. 2 and 3.

FIG. 5 shows an enlarged view of treated tonsils.

### DETAILED DESCRIPTION OF THE INVENTION

According to the invention tonsils can be treated by using an applicator 1 in a bipolar design (see FIG. 1). This means that no neutral electrode (i.e. no separate return electrode) is necessary.

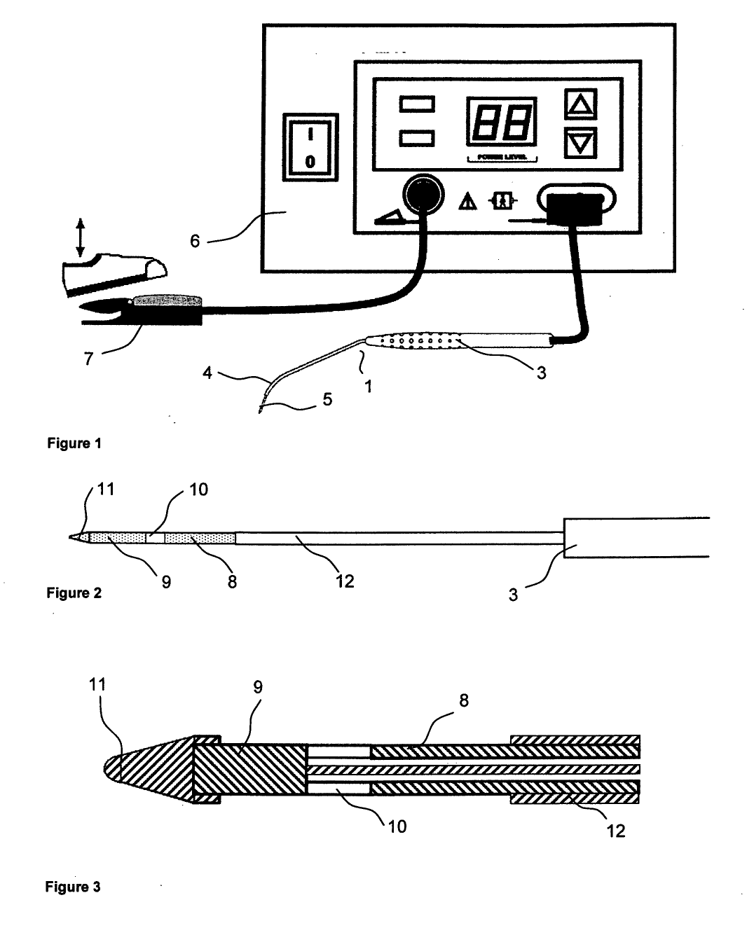

The applicator 1 used for carrying out the inventive method comprises a hand portion 3 and a shaft 4 which is connected with the hand portion 3. The shaft 4 has a distal portion 5 carrying at least two electrodes 8 and 9 which are arranged on the shaft 4 and which are spaced apart from each other in axial direction of the shaft 4. The electrode dose to the hand portion is a proximal electrode 8 and the electrode further to the hand portion is a distal electrode 9 (see FIGS. 2 and 3). A proximal part of the proximal electrode 8 is insulated by an insulation tube 12. Both electrodes 8, 9 are electrically insulated from each other by an insulator 10. The distal electrode 9 has a conical or trocar shape tip 11 to penetrate the treated tonsil tissue 14 easily. The electrodes diameter is between 1 to 1.5 mm, preferably 1.3 mm.

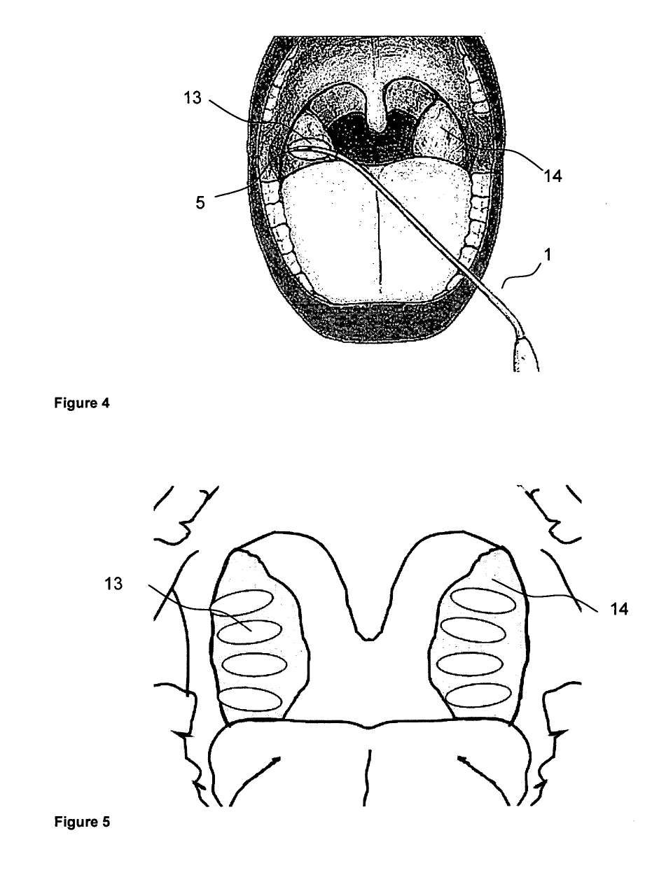

For a treatment of Hypertrophic Palatine Tonsils 14 the electrodes 5 of the applicator 1 are connected to different poles of a high frequency current generator 6 (see FIG. 4).

During the treatment the high frequency current (provided by a high frequency generator at an output frequency range of 300 to 500 kHz) flows through the tissue between the distal and the proximal electrode 8 and 9 at the tip 11 of the applicator 1 and heats up the tissue 13 surrounding the electrodes (thermal lesion) (see FIGS. 4 and 5).

The delivery of the high frequency current to the applicator 1 and thus the duration of treatment is controlled by a pedal 7.

The treatment of Hypertrophic Palatine Tonsils 14 is to be repeated multiple times in order achieve a plurality of lesions (see FIG. 5).

The method for treatment of Hypertrophic Palatine Tonsils comprises the steps of:
1) connecting the applicator with a high frequency generator and a pedal;
2) setting the power output of the high, frequency generator to a value between 1 to 25 W, preferably 7 watts;
3) inserting the distal applicator tip into a tonsil to be treated until the insulation tube on the applicator shaft contacts the tonsil tissue and both electrodes are completely covered by the tonsil tissue to be treated;
4) activating the high frequency current in order to create a thermal lesion until the high frequency generator indicates the end of treatment by a pulsed acoustic signal, meaning that the dehydration of the treated tonsil tissue has been reached;
5) retracting the applicator tip from the tonsil; and
6) repeating the steps of inserting, activating and retracting at different positions of the tonsil until a major part of the tonsil tissue is coagulated.

The exact number of lesions 13 (treated tonsil tissue) depends on the size of the tonsil 14, The average number of punctures per tonsil is 4 to 5.

If the tonsil size is small, penetration of the underlying tonsil fibrous capsule should be avoided. To prevent damage to underlying vessels and nerves the thermal lesion should not be positioned too dose to the tonsil fossa bed and tonsil pillars. The distance between the thermal lesion and the critical structures can be increased by pulling the tonsils median.

The resulting thermal lesions 13 (volumes of treated tonsil tissue) should be arranged to each other in a distance as shown in FIG. 5.

Finally, the treatment comprises the step of letting die coagulated tissue 13 be resorbed by body-own processes. This results in a volume reduction of the enlarged tonsils. The average volume shrinkage is about 30-50%.

The method may further comprise one or more of the following steps of Pain reduction, Disinfection and Anesthesia:
for pre-operative pain reduction: administrating 1 tablet (50 mg) Diclophenac;
for superficial disinfection of the tonsils 14: administrating Hexetidin
for superficial anesthesia: administrating Lidocaine spray 2%
for local anesthesia of the tonsil 14: injecting 4 ml Lidocaine 2% with Adrenalin per tonsil into each tonsil;
for peri-operative antibiotic prophylaxis: administrating 2×500 mg Cefuroxim per day for one week; and
For post-operative pain reduction: administrating diclophenac 2-3 tablets (50 mg) per day.

The many features and advantages of the invention are apparent from the above description. Numerous modifications and variations will readily occur to those skilled in the art. Since such modifications are possible, the invention is not to be limited to the exact construction and operation illustrated and described. Rather, the present invention should be limited only by the following claims.

1. Method for treating Hypertrophic Palatine Tonsils comprising the steps of:
a) pre-operative pain reduction,
b) superficial disinfection of the tonsils,
c) local anesthesia of the tonsil or general anesthesia,
d) treating the tonsils using a bipolar applicator having two electrodes coaxially arranged on a common shaft and spaced apart from each other in the longitudinal direction of the shaft,
e) the use of the applicator comprises the steps of:
i) connecting the applicator with a high frequency generator,
ii) setting the power Output of the high frequency generator to a value between 1 to 25 W, preferably 7 watts,
iii) inserting the distal applicator tip into a tonsil to be treated until the Insulation tube on the applicator shaft contacts the tonsil tissue and both electrodes are completely covered by the tonsil tissue,
iv) activating the high frequency current in order to create a thermal lesion until the high frequency generator indicates the end of treatment by a pulsed acoustic signal, meaning that the dehydration of the tonsil tissue has been reached,
v) retracting the applicator tip from the tonsil, and
vi) repeating the steps of Inserting, Activating and Retracting at different positions of the tonsil until a major part of the tonsil tissue is coagulated,
f) peri-operative antibiotic prophylaxis, and
g) post-operative pain reduction.

2. The method according to claim 1, wherein the step of pre-operative pain reduction includes administrating 1 tablet (50 mg) Diclophenac.

3. The method according to claim 1, wherein the step of superficial disinfection of the tonsils includes administrating Hexetidin.

4. The method according to claim 1, wherein the step of superficial anesthesia includes administrating Lidocaine spray having a concentration of 2%.

5. The method according to claim 1, wherein the step of local anesthesia of the tonsil includes injecting 4 ml Lidocaine of 2% concentration with Adrenalin per tonsil into each tonsil.

6. The method according to claim 1, wherein the step of peri-operative antibiotic prophylaxis includes administrating 2×500 mg Cefuroxim per day for one week; and

7. The method according to claim 1, wherein the step of post-operative pain reduction includes administrating didophenac 2-3 tablets (50 mg) per day.
